(19) United States
(12) Patent Application Publication
(10) Pub. No.: US 2011/0033506 A1
(43) Pub. Date: Feb. 10, 2011

(54) COMBINATION DOSAGE FORM OF LOW-DOSE MODAFINIL AND LOW-DOSE SILDENAFIL

(76) Inventors: Adel Penhasi, Holon (IL); Janina Gauchman, Givon Ha Hadasha (IL); Amanda Shauli, Zichron Yaacov (IL); Marina Ruderman, Zichron Yaakov (IL); Avi Avramoff, Haifa (IL)

Correspondence Address: DR. D. GRAESER LTD., 9003 FLORIN WAY, UPPER MARLBORO, MD, 20772, US

(21) Appl. No.: 12/865,727

(22) PCT Filed: Feb. 8, 2009
(86) PCT No.: PCT/IL09/00146

§ 371 Date: Oct. 19, 2010

Related U.S. Application Data
(60) Provisional application No. 61/006,956, filed on Feb. 8, 2008.

(51) Int. Cl. A61K 9/00; A61K 31/165; A61K 31/496; A61P 25/00; A61P 29/00

(52) U.S. Cl. 424/400; 514/618; 514/252.16

(57) ABSTRACT

Modified release formulations comprising at least one of modafinil and sildenafil, and combinations thereof, and a time-controlled delivery system coating.

### FIELD OF THE INVENTION

The present invention relates to modified release formulations comprising modafinil and/or sildenafil.

### BACKGROUND OF THE INVENTION

Sleep disorders are a broad range of conditions arising from many causes, including dysfunctional sleep mechanisms, abnormalities in physiological functions during sleep, abnormalities of the biological clock, and sleep disturbances that are induced by factors extrinsic to the sleep process, including jet lag and shift work.

Modafinil (C₁₅H₁₅NO₂S), of chemical structure:

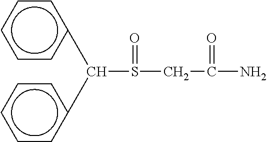

is a psychostimulant which enhances wakefulness, attention capacity and vigilance.

Modafinil induces wakefulness in part by its action in the anterior hypothalamus. Its dopamine-releasing action in the nucleus accumbens is weak and dose-dependent; the likelihood of a euphoric response ('abuse potential'), dose-escalation and tolerance is thus apparently small.

Modafinil has central alpha 1-adrenergic agonist effects and inhibits the reuptake of noradrenaline by the noradrenergic terminals on sleep-promoting neurons of ventrolateral preoptic nucleus. Modafinil is also able to increase excitatory glutamatergic transmission, which reduces local GABAergic transmission, thereby diminishing GABA(A) receptor signaling on the mesolimbic dopamine terminals.

Modafinil is approved in the United States for the treatment of narcolepsy, a neurological disorder characterized by excessive sleepiness, cataplexy, sleep paralysis, hypnogogic hallucinations, and an abnormal tendency to pass directly from wakefulness into rapid eye-movement (REM) sleep. Narcolepsy is caused by dysfunction of a family of wakefulness-promoting and sleep-suppressing peptides, the orexins. Orexin neurons, which are found exclusively in the lateral hypothalamic area, are activated by modafinil. Orexinergic fibers project to the entire central nervous system. Narcoleptics suffer profound disturbances in normal sleeping patterns and variable degrees of depression. These symptoms can be reversed with modafinil.

Modafinil is also approved in the United States for the treatment of obstructive sleep apnea/hypopnea and sleepiness in shift workers.

Outside of the U.S., modafinil is also indicated for idiopathic hypersomnia (all forms of excessive daytime sleepiness of unknown cause).

Modafinil is also considered to be useful in the treatment of jet lag, which is a sleep disturbance induced by a major rapid shift in environmental time during travel to a new time zone. Modafinil has further suggested as being useful in the treatment of Alzheimer's disease; depression; attention-deficit disorder (ADHD); myotonic dystrophy; multiple sclerosis-induced fatigue; post-anaesthesia grogginess; cognitive impairment in schizophrenia; spasticity associated with cerebral palsy, age-related memory decline; idiopathic hypersomnia; methamphetamine abuse; apathy in the elderly; cancer-associated fatigue and opioid-induced sedation; fatigue in Charcot-Marie-Tooth Disease and everyday cat-napping.

The recommended dose of Modafinil is 200 mg given once a day. Side effects associated with such doses commonly include headache, nausea, nervousness, rhinitis, diarrhea, back pain, anxiety, insomnia, dizziness, and dyspepsia.

Sildenafil (Viagra®) 1-[[3-(6,7-dihydro-1-methyl-7-oxo-3-propyl-1H-pyrazolo[4,3-d]pyrimidin-5-yl)-4-ethoxyphenyl]sulfonyl]-4-methylpiperazine of structural formula:

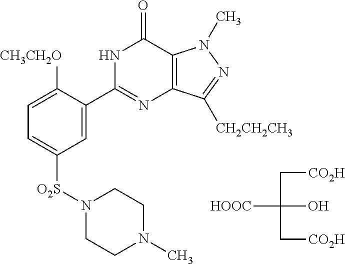

is a selective inhibitor of cyclic guanosine monophosphate (cGMP)-specific phosphodiesterase type 5, commonly used to treat erectile dysfunction.

Sildenafil was recently shown to be effective in re-establishing disrupted circadian rhythms, or “jet lag”, in hamsters (Sildenafil accelerates reentrainment of circadian rhythms after advancing light schedules; Agostino et al, PNAS, Jun. 5, 2007, vol 104, no 23, pp 9834-9839). PCT Application WO 2001/062290 disclosed the use of sildenafil to induce sleep. Thus the correct role and impact of sildenafil on the sleep process is not known, due the different contradictory study results.

Sildenafil is currently supplied as tablets of 25 mg, 50 mg and 100 mg of sildenafil for oral administration. Sildenafil side-effects include headaches, dependency and vision abnormalities. Drowsiness has been also reported as a clinical effect upon use of sildenafil (Journal of toxicology and environmental health. Part A, (2006 Mar.) Vol. 69, No. 6, pp. 497-503). It is considered that drowsiness and sleepiness are probably indirect result of the lowering of blood pressure.

### SUMMARY OF THE INVENTION

The background art does not teach or suggest modified release formulations for at least one of a wakefulness inducing agent (such as modafinil) and a phosphodiesterase type 5 inhibitor (such as sildenafil), and combinations thereof, particularly for increasing wakefulness, and treatment of sleepiness, jetlag, and sleep disorders associated with excessive sleepiness.

The present invention overcomes the deficiencies of the background art by providing delayed onset, slow release formulations of at least one of a wakefulness inducing agent, such as modafinil, and a phosphodiesterase type 5 inhibitor, such as sildenafil, or pharmaceutically acceptable salts thereof, as well as a composition comprising a combination dosage form of a low-dose wakefulness inducing agent, and a low-dose phosphodiesterase type 5 inhibitor optionally and preferably for increasing wakefulness, and treatment of sleepiness, jetlag, and sleep disorders associated with excessive sleepiness. The combination dosage form enables low doses of one or both of modafinil and sildenafil to be used, thereby potentially avoiding problematic or even dangerous side effects.

According to some embodiments of the present invention, there is provided a composition comprising a core and a time-controlled delivery system (TCDS) coating. The core comprises at least one of a wakefulness-inducing agent and a phosphodiesterase type 5 inhibitor as active ingredient.

According to some embodiments, the wakefulness-inducing agent is at least one of modafinil, armodafinil, a xanthine derivative, caffeine, a centrally acting sympathomimetic, or a combination thereof.

According to some embodiments, the phosphodiesterase type 5 inhibitor is at least one of sildenafil, tadalafil, vardenafil or a combination thereof.

According to some embodiments, the core further comprises at least one of a filler, a binder, a disintegrant, a release controlling agent, a glidant and a lubricant.

According to some embodiments, the TCDS coating comprises at least one of a pH dependent coating film; a combination of two or more water insoluble polymers; a combination of at least one swellable polymer and at least one water insoluble polymer; a flexible but non-soluble coating; a swellable coating; and a water permeable coating.

The pH dependent coating polymer of the TCDS coating optionally comprises at least one of a hydroxypropylmethyl cellulose phthalate, polyvinyl acetate phthalate, cellulose acetate phthalate, hydroxypropylmethyl cellulose acetate succinate, poly(methacrylic acid, methyl methacrylate) 1:1 and poly(methacrylic acid, ethyl acrylate) 1:1, alginic acid, and sodium alginate.

According to a preferred embodiment, the polymer of the TCDS coating comprises Eudragit™.

The water insoluble polymer of the TCDS coating optionally comprises at least one of a polymethylaminoethylacrylate/ethylmethacrylate copolymer; an ethylmethacrylate/chlorotrimethylammoniumethyl methacrylate copolymer; a dimethylaminoethylmethacrylate/methylmethacrylate and butylmethacrylate copolymer; a copolymer based on neutral methacrylic acid esters and dimethylaminoethyl methacrylate esters; an ethylacrylate and methylacrylate/ethylmethacrylate and methyl methylacrylate copolymer; ethylcellulose; shellac; zein; a wax; paraffin; cellulose acetate; cellulose propionate; cellulose acetate; propionate; cellulose acetate butyrate; cellulose acetate phthalate; cellulose triacetate; poly(methyl methacrylate); poly(ethylmethacrylate); poly (butyl methacrylate); poly(isobutyl methacrylate); poly(hexyl methacrylate); poly(isodecyl methacrylate); poly(lauryl methacrylate); poly(phenyl methacrylate); poly(methylacrylate); poly(isopropyl acrylate); poly(isobutyl acrylate); poly(octadecyl acrylate); poly (ethylene); poly(ethylene oxide); poly(ethyleneterephthalate); poly(vinyl isobutyl ether); poly(vinyl acetate); poly(vinyl chloride) and polyurethane, or mixtures thereof.

According to a preferred embodiment, the water insoluble polymer comprises ethylcellulose.

According to some embodiments, the TCDS coating further comprises a diffusion controlling agent.

According to some embodiments, the TCDS coating further comprises a plasticizer.

According to some embodiments, the composition further comprises a protective subcoating layer between the core and the TCDS coating.

According to some embodiments, the composition further comprises an enteric coating on the TCDS coating.

According to some embodiments, the composition further comprises a protective subcoating layer between the TCDS coating and the enteric coating.

According to some embodiments, the protective subcoating layer comprises a coating polymer selected from the group consisting of polyvinyl pyrrolidone, hydroxypropyl cellulose, hydroxypropyl methylcellulose, methyl cellulose, ethylcellulose, and gelatin polyethylene oxide.

According to some embodiments, the coating polymer comprises polyvinyl pyrrolidone.

According to some embodiments, the protective subcoating layer further comprises a glidant. Optionally and preferably, the glidant comprises at least one of microcrystalline cellulose, talc, colloidal hydrated aluminum silicate, and silicon dioxide, or combinations thereof.

According to some embodiments, the composition further comprises an acidic coating layer between the core and the TCDS layer. The acidic coating layer optionally and preferably comprises an acidifying agent and a coating polymer.

According to a preferred embodiment, the acidifying agent comprises citric acid and the coating polymer comprises Povidone.

According to some embodiments, the acidic coating layer further comprises a glidant.

According to some embodiments, the composition of the present invention comprises a wakefulness-inducing agent and a phosphodiesterase type 5 inhibitor as active ingredients.

According to a preferred embodiment, the wakefulness-inducing agent comprises modafinil or pharmaceutically acceptable salts thereof, and the phosphodiesterase type 5 inhibitor comprises sildenafil, or pharmaceutically acceptable salts thereof.

Optionally and preferably, one or both of modafinil and sildenafil are present in a lower than normal dose. Further optionally and preferably, the lower than normal dose is a sub-therapeutic dose.

According to some embodiments, composition of the present invention comprises an active core. Alternatively or additionally, the core may comprise an active coating layer.

According to some embodiments, the active core comprises a first of modafinil or sildenafil, and the active coating layer comprises a second of modafinil or sildenafil.

According to some embodiments, sildenafil is released during or before the period of desired sleeping, while said modafinil is released at or near or toward the period of desired wakefulness; or alternatively wherein said sildenafil and said modafinil are released simultaneously or substantially simultaneously and/or at or near or toward the period of desired wakefulness.

According to some embodiments, there is provided the use of any of the compositions of the present invention for increasing wakefulness, treatment or prevention of sleepiness, treatment or prevention of jetlag, treatment or prevention of sleep disorders associated with excessive sleepiness, and reduction or prevention of sleepiness throughout a desired period.

The sleep disorder which may be treated or prevented by the compositions of the present invention may be one or more of narcolepsy, obstructive sleep apnea/hypopnea and sleepiness in shift workers, idiopathic hypersomnia, multiple sclerosis-induced fatigue; post-anaesthesia grogginess; opioid-induced sedation; and fatigue in Charcot-Marie-Tooth Disease.

According to some embodiments, the composition of the present invention further comprises an additional active ingredient, such as, for example, an agent for reducing or eliminating headache, such as an NSAID or paracetamol.

According to some embodiments, the present invention provides a synergistic dosage form for treating sleep related disorders, comprising a synergistic combination of a PDE5 inhibitor and a wakefulness inducing agent.

### BRIEF DESCRIPTION OF THE DRAWINGS

The invention is herein described, by way of example only, with reference to the accompanying drawings, wherein:

FIG. 1 shows dissolution of uncoated modafinil cores (1-4) in 0.1 N HCL (Average Release of 6 tablets);

FIG. 2 shows dissolution of uncoated modafinil cores (5 and 6) in phosphate buffer pH=6.8 (Average Release of 6 tablets);

FIG. 3 shows dissolution of TCDS coated modafinil core 1 in phosphate buffer pH=6.8 (Average Release of 6 tablets);

FIG. 4 shows dissolution of TCDS coated modafinil cores 6A, 6B and 6C in phosphate buffer=6.8;

FIG. 5 shows dissolution of modafinil cores 4 and 5 coated with protective layer and TCDS layer in phosphate buffer=6.8;

FIG. 6 shows dissolution of uncoated sildenafil cores in 0.1 N HCl or phosphate buffer=6.8;

FIG. 7 shows dissolution of TCDS coated sildenafil cores 7A-1 and 7A-2 in buffer phosphate=6.8;

FIG. 8 shows dissolution of sildenafil cores coated with acid layer and TCDS layer (8A, 8B, 9A, 9B) in acetate buffer=5.8;

FIG. 9 shows dissolution of TCDS and enteric coated layer sildenafil cores (8C and 9C) in acetate buffer=5.8;

FIG. 10 shows dissolution of multilayer coated sildenafil core 9D in phosphate buffer=6.8; and

FIG. 11 shows dissolution of combination modafinil and sildenafil dosage form in phosphate buffer=6.8.

### DESCRIPTION OF THE PREFERRED EMBODIMENTS

The present invention provides delayed onset, slow release forms of at least one of modafinil and sildenafil, as well as a composition comprising a combination dosage form of low-dose wakefulness inducing agent, such as modafinil, and low-dose phosphodiesterase type 5 inhibitor, such as sildenafil, or pharmaceutically acceptable salts thereof, optionally and preferably for increasing wakefulness, and treatment of sleepiness, jetlag, and sleep disorders associated with excessive sleepiness.

Controlled release formulations for oral administration of drugs are beneficial for a number of reasons. For example, they enable the patient to ingest the formulation less frequently, which may lead to increased patient compliance with the dosing regimen. They may also result in fewer side effects, as peaks and troughs of the level of the drug in the bloodstream of the patient may be decreased, leading to a more even drug level in the blood over a period of time. Such formulations may also provide a longer plateau concentration of the drug in the blood. The size and frequency of dosing is determined by the pharmacodynamic and pharmacokinetic properties of the drug. The slower the rate of absorption, the less the blood concentrations fluctuate within a dosing interval. This enables higher doses to be given less frequently. For drugs with relatively short half-lives, the use of modified-release products may maintain therapeutic concentrations over prolonged periods.

Modified release products may also be very effective for combinations of two or more drugs, in which each drug provides a particular functionality or activity, such that the modified release formulation may then control the separate release of each drug.

US Patent Application No. 20050095294 discusses modified release of modafinil with two pulses of the drug. However, it does not discuss or suggest delayed onset, slow release dosage forms of modafinil. Combinations with anti-depressants are discussed but not with other types of drugs.

According to some embodiments, the composition of the present invention comprises a core comprising at least one of a wakefulness-inducing agent and a phosphodiesterase type 5 inhibitor and a time-controlled delivery system (TCDS) coating.

According to some embodiments, the coating may comprise a protective subcoating layer beneath the TCDS layer.

According to some embodiments, the coating may comprise an acidic inner coating layer beneath the TCDS coating layer.

According to some embodiments, the coating may comprise an enteric coating layer. Optionally, the coating may further comprise a protective coating layer beneath the enteric coating layer.

### Active Ingredients

The active ingredient comprises at least one of modafinil and sildenafil, including combinations thereof.

According to some embodiments, drugs belonging to the same classes as modafinil and sildenafil may optionally be used in place of or in addition to one or more of these active ingredients, including phosphodiesterase type 5 inhibitors generally and agents for improving wakefulness generally.

Optionally and preferably, the phosphodiesterase type 5 inhibitor is selected from the group consisting of sildenafil, tadalafil, vardenafil or a combination thereof.

Also optionally and preferably, the wakefulness inducing agent is selected from the group consisting of modafinil or armodafinil, xanthine derivatives such as caffeine, centrally acting sympathomimetics such as amphetamines, methylphenidate, or a combination thereof.

### Core

The core comprises at least one of a wakefulness-inducing agent and a phosphodiesterase type 5 inhibitor as active ingredients.

According to some embodiments, the core is an active core comprising the active ingredient(s).

The active core could be prepared in a number of different ways which are known in the art. For example, the active core could be formed by granulating the active ingredient with suitable excipients, and compressing the granulate into tablets.

Alternatively, the composition could include a neutral core, such as a sugar bead which does not contain an active ingredient, over which the active ingredients are coated, preferably with a suitable adhesive polymer. The neutral core may optionally comprise at least one of a non-pareil, a bead, a seed, a granule, and a pellet.

Further optionally, the composition may comprise an active core comprising a first of the wakefulness-inducing agent and the phosphodiesterase type 5 inhibitor, and this active core is coated with an active layer comprising the second of the wakefulness-inducing agent and the phosphodiesterase type 5 inhibitor.

According to some embodiments, the core further comprises at least one of a filler, a binder, and a disintegrant.

Optionally and preferably, the filler is selected from the group consisting of microcrystalline cellulose, starch, lactitol, lactose, a suitable inorganic calcium salt, sucrose, or a combination thereof. More preferably the filler comprises lactose monohydrate and/or microcrystalline cellulose.

The binder is optionally and preferably selected from the group consisting of Povidone (PVP: polyvinyl pyrrolidone), low molecular weight HPC (hydroxypropyl cellulose), low molecular weight HPMC (hydroxypropyl methylcellulose), low molecular weight carboxy methyl cellulose, ethylcellulose, gelatin polyethylene oxide, acacia, dextrin, magnesium aluminum silicate, starch, and polymethacrylates. Preferably, the binder is Povidone.

Optionally and preferably, the disintegrant is selected from the group consisting of croscarmellose sodium, crospovidone (cross-linked polyvinyl pyrolidone) sodium carboxymethyl starch (sodium starch glycolate), cross-linked sodium carboxymethyl cellulose (Croscarmellose), pregelatinized starch (starch 1500), microcrystalline starch, water insoluble starch, calcium carboxymethyl cellulose, magnesium aluminum silicate and a combination thereof. More preferably, the disintegrating agent comprises one or more of croscarmellose sodium, starch and crospovidone.

The mechanism of disintegration is based on swelling, wicking, and deformation of the disintegrants. Some commercial superdisintegrants for use in the present invention include but are not limited to Ac-Di-Sol, Primojel, Explotab, and Crospovidone. The core of the present invention optionally and preferably includes a wicking agent in addition to or as an alternative to a disintegrant.

According to some embodiments, the core further comprises one or more of a release controlling agent, a glidant and a lubricant.

The release controlling agent may comprise, for example, Tween or suitable hydrophilic gelling polymers including but not limited to cellulosic polymers, such as methylcellulose, carboxymethylcellulose, hydroxypropylcellulose, hydroxypropylmethylcellulose, hydroxyethylcellulose, and the like; vinyl polymers, such as polyvinylpyrrolidone, polyvinyl alcohol, and the like; acrylic polymers and copolymers, such as acrylic acid polymer, methacrylic acid copolymers, ethyl acrylate-methyl methacrylate copolymers, natural and synthetic gums, such as guar gum, arabic gum, xanthan gum, gelatin, collagen, proteins, polysaccharides, such as pectin, pectic acid, alginic acid, sodium alginate, polyaminoacids, polyalcohols, polyglycols; and the like; and mixtures thereof.

The polysaccharide may optionally be a cross-linked polysaccharide, such as a cross-linked polysaccharide selected from the group consisting of insoluble metal salts or cross-linked derivatives of alginate, pectin, xanthan gum, guar gum, tragacanth gum, and locust bean gum, carrageenan, metal salts thereof, and covalently cross-linked derivatives thereof.

The cellulosic polymer may optionally be a modified cellulose, such as a modified cellulose selected from the group consisting of cross-linked derivatives of hydroxypropylcellulose, hydroxypropylmethylcellulose, hydroxyethylcellulose, methylcellulose, carboxymethylcellulose, and metal salts of carboxymethylcellulose.

According to other optional but preferred embodiments of the present invention, the core is a delayed onset, slow release core in which the time and rate of release are controlled through the amount of release controlling agent.

According to some embodiments, the glidant is selected from the group consisting of microcrystalline cellulose, talc, colloidal hydrated aluminum silicate (bentonit), or silicon dioxide. Preferably, the glidant comprises silicon dioxide.

According to some embodiments, the lubricant is selected from the group consisting of stearate salts; stearic acid, corola oil, glyceryl palmitostearate, hydrogenated vegetable oil, magnesium oxide, mineral oil, poloxamer, polyethylene glycole, polyvinyl alcohol, sodium benzoate, talc, sodium stearyl fumarate, compritol (glycerol behenate), and sodium lauryl sulfate (SLS) or a combination thereof. Most preferably, the lubricant is magnesium stearate.

Optionally, the core further comprises at least one of an absorption enhancer, and a hardness enhancing agent.

Examples of suitable absorption enhancers include bile salts, aromatic alcohols, medium chain fatty acids, medium chain monoglycerides, phospholipids, fatty acids, surface active agents, EDTA and related salts.

Examples of suitable hardness enhancing agents include, without limitation, sorbitol, microcrystalline cellulose, mannitol, lactose, and starch.

Optionally and preferably, the core also includes a stabilizer. More preferably, the stabilizer comprises at least one of butyl hydroxyanisole, ascorbic acid and citric acid.

Also alternatively or additionally, the core may optionally further comprise a flow regulating agent. Preferably, the flow regulating agent includes at least one of colloidal silicon dioxide and aluminum silicate. Most preferably, the flow regulating agent is colloidal silicon dioxide.

More preferably, the core is in the form of one of a tablet, pellets, microparticles, agglomerate, and capsule.

### Coating

The core is preferably coated with a Time Controlled Delivery System (TCDS) coating.

The TCDS coating is optionally and preferably selected from the group consisting of a pH dependent coating film (featuring a pH dependent polymer); a combination of two or more water insoluble polymers; a combination of at least one swellable polymer and at least one water insoluble polymer; a flexible but non-soluble coating; a swellable coating, which may optionally be a dry coating featuring a gel forming material; and a water permeable coating.

According to some embodiments, the TCDS coating comprises a coating polymer and a diffusion controlling agent. Optionally and preferably, the polymer is a water insoluble polymer, such as, for example, ethylcellulose or a pH dependent polymer, such as, for example, Eudragit. The TCDS layer optionally and preferably further comprises a diffusion controlling agent, such as, for example, microcrystalline cellulose.

Examples of suitable pH dependent polymers of the TCDS are preferably selected from the group consisting of a hydroxypropylmethyl cellulose phthalate, polyvinyl acetate phthalate, cellulose acetate phthalate, hydroxypropylmethyl cellulose acetate succinate, poly(methacrylic acid, methyl methacrylate) 1:1 and poly(methacrylic acid, ethyl acrylate) 1:1, alginic acid, and sodium alginate.

A suitable TCDS coating polymer can be from Eudragit™ polymers series (available from Rohm Pharma) which are polymeric lacquer substances based on acrylates and/or methacrylates. Suitable polymers which are slightly permeable to water, and exhibit a pH-dependent permeability include, but are not limited to, Eudragit™ L, and Eudragit™ S. Eudragit™ L is an anionic polymer synthesized from methacrylic acid and methacrylic acid methyl ester. It is insoluble in acids and pure water. It becomes soluble in neutral to weakly alkaline conditions. The permeability of Eudragit™ L is pH dependent. Above pH 5.0, the polymer becomes increasingly permeable.

According to some embodiments, the polymeric material comprises methacrylic acid co-polymers, ammonio methacrylate co-polymers, or a mixture thereof. Methacrylic acid co-polymers such as Eudragit™ S and Eudragit™ L (Rohm Pharma) are suitable for use in the delayed onset, modified release formulations of the present invention, these polymers are gastro-resistant and entero-soluble polymers, providing a delay in onset of the release depending on the pH, the type of the polymer (Eudragit L or Eudragit S) and the thickness of the film coat.

The films of Methacrylic acid co-polymers such as Eudragit™ S and Eudragit™ L are insoluble in pure water and diluted acids. They dissolve at higher pH values, depending on their content of carboxylic acid. Eudragit™ S and Eudragit™ L can be used as single components in the coating of the formulation of the present invention or in combination in any ratio. By using a combination of the polymers, the polymeric material may exhibit a solubility at a pH between the pHs at which Eudragit™ L and Eudragit™ S are separately soluble.

Optionally, the outer coating further comprises a plasticizer. More preferably, the plasticizer includes at least one of dibutyl sebacate, polyethylene glycol and polypropylene glycol, dibutyl phthalate, diethyl phthalate, triethyl citrate, tributyl citrate, acetylated monoglyceride, acetyl tributyl citrate, triacetin, dimethyl phthalate, benzyl benzoate, butyl and/or glycol esters of fatty acids, refined mineral oils, oleic acid, castor oil, corn oil, camphor, glycerol and sorbitol or a combination thereof.

In another embodiment according to the present invention the delayed onset, controlled release formulation may comprise a release controlling core formulation and an outer coating, optionally comprising a combination of a water soluble polymer and/or a water swellable hydrophilic polymer and a water insoluble polymer. In this manner, where the film coating formulation features a combination of at least a water soluble polymer and at least a water insoluble polymer, it is possible to provide a delay time prior to the release of the active material, depending on the relative content (weight fraction) of the water soluble polymer in the outer coating, the thickness of the outer film coat, and the nature of the polymers present in the outer film coat. Without wishing to be limited by a single hypothesis, upon exposure of the formulation to the gastrointestinal fluids, the water soluble polymer starts to dissolve, leaving channels that allow penetration of the gastrointestinal fluids into the core, which may eventually lead to a slow disintegration of the core and thus a slow release of the active material.

A non-limiting, illustrative example according to this embodiment may be based on a core which can be formulated as described above for the previous embodiment, and a TCDS coating comprising a totally water soluble polymer and a water insoluble polymer. Without wishing to be limited by a single hypothesis, coating disintegration may be due to increased osmotic pressure in the core. Suitable water-soluble polymers include, but are not limited to, polyvinyl alcohol, polyvinylpyrrolidone (PVP), copolyvidone (a (6:4) copolymer of a chain-structured vinyl pyrrolidone and vinyl acetate), methylcellulose, hydroxypropylcellulose, hydroxypropylmethyl cellulose, polyethylene glycol, carboxymethyl cellulose (sodium salt), hydroxyethyl cellulose, a water soluble gum, polysaccharide and/or mixtures thereof.

Suitable water insoluble polymers of the TCDS coating are selected from the group consisting of a polymethylaminoethylacrylate/ethylmethacrylate copolymer, the copolymer being based on acrylic and methacrylic acid esters with a low content of quaternary ammonium groups, wherein the molar ratio of the ammonium groups to the remaining neutral (meth)acrylic acid esters is approximately 1:20, the polymer corresponding to USP/NF “Ammonio Methacrylate Copolymer Type A”, an ethylmethacrylate/chlorotrimethylammoniumethyl methacrylate copolymer, the copolymer based on acrylic and methacrylic acid esters with a low content of quaternary ammonium groups wherein the molar ratio of the ammonium groups to the remaining neutral (meth)acrylic acid esters is 1:40, the polymer corresponding to USP/NF “Ammonio Methacrylate Copolymer Type B”, a dimethylaminoethylmethacrylate/methylmethacrylate and butylmethacrylate copolymer, a copolymer based on neutral methacrylic acid esters and dimethylaminoethyl methacrylate esters wherein the polymer is cationic in the presence of acids, an ethylacrylate and methylacrylate/ethylmethacrylate and methyl methylacrylate copolymer, the copolymer being a neutral copolymer based on neutral methacrylic acid and acrylic acid esters, ethylcellulose, shellac, zein, and waxes, paraffin, cellulose acetate, cellulose propionate, cellulose acetate propionate, cellulose acetate butyrate, cellulose acetate phthalate, cellulose triacetate, poly(methyl methacrylate), poly(ethylmethacrylate), poly(butyl methacrylate), poly(isobutyl methacrylate), and poly(hexyl methacrylate), poly (isodecyl methacrylate), poly(lauryl methacrylate), poly(phenyl methacrylate), poly (methylacrylate), poly(isopropyl acrylate), poly(isobutyl acrylate) poly(octadecyl acrylate), poly(ethylene), poly(ethylene) low density, poly(ethylene) high density, poly(ethylene oxide), poly(ethyleneterephthalate), poly(vinyl isobutyl ether), poly(vinyl acetate), poly(vinyl chloride) and polyurethane, and/or mixtures thereof. More preferably, the water insoluble polymer is ethylcellulose.

### Enteric Coating

Optionally, the formulation may further comprise an enteric coating disposed on the outer coating. Enteric coatings may optionally be selected from any of the pH dependent polymers described above.

The enteric coating layer may optionally and preferably further comprise a plasticizer, such as triethyl citrate; and a glidant, such as talc.

### Protective Coating

According to some embodiments, a protective subcoating is provided between the core and the TCDS coating and/or between the TCDS layer and the enteric coating layer. The protective coating preferably comprises a coating polymer, more preferably comprising one or more of Povidone (PVP: polyvinyl pyrrolidone), low molecular weight HPC (hydroxypropyl cellulose), low molecular weight HPMC (hydroxypropyl methylcellulose), low molecular weight carboxy methyl cellulose, ethylcellulose, and gelatin polyethylene oxide. Preferably, the coating polymer comprises Povidone.

The protective coating also preferably comprises a glidant, more preferably comprising one or more of microcrystalline cellulose, talc, colloidal hydrated aluminum silicate (bentonit), or colloidal silicon dioxide (aerosil).

### Acidic Layer

According to some embodiments, an acid layer is optionally provided as a first inner coating layer, between the core and the TCDS layer. The acidic layer preferably comprises a coating polymer, such as, for example, Povidone, and an acidifying agent that can cause the dissolution of the water insoluble components of the outer coating (TCDS-based coating) and act as a drug release enhancer. Upon absorption of liquid, channels are formed connecting the outer TCDS layer and the acidifying agent-containing inner layer leading to the dissolution of the acid inner coating layer, whereby the agents contained therein are released and cause the dissolution of the outer TCDS coating.

The acid layer optionally and preferably further comprises a glidant.

According to some embodiments, the acid layer comprises citric acid as drug release enhancer, Povidone K 30 as a coating polymer and silica colloidal anhydrous as a glidant

### Combination Dosage Forms of Modafinil and Sildenafil

According to some embodiments, the present invention provides a combination dosage form comprising modafinil and sildenafil.

Optionally, the dosage form comprises at least one tablet comprising modafinil and at least one tablet comprising sildenafil, in a capsule, such as a hard gelatin capsule. Each of the tablets may optionally comprise any of the modified release formulations described above for modafinil and sildenafil.

Alternatively, the active ingredients may be combined in a single dosage form, such as in an active core; in an active layer on a neutral core; or in a tablet wherein a first of the active ingredients is present in an active core and a second of the active ingredients is present in an active coating.

The combination dosage form is capable of providing a therapeutically effective amount of sildenafil and modafinil, a pharmaceutically acceptable salt or ester thereof or an active form thereof to a subject, for an extended period of time after the start of release, preferably at a lower dose of one or both of the active ingredients.

Without wishing to be limited by a single hypothesis, the combination dosage form of the present invention provide a synergistic effect for the treatment of sleep disorders through a combination of sildenafil and modafinil, which was not taught or suggested by the background art. Synergism may also optionally be seen through faster onset of a desired treatment effect.

### Therapeutic Uses

Any of the dosage form of the present invention may be used for increasing wakefulness, and treatment or prevention of sleepiness, jetlag, and sleep disorders associated with excessive sleepiness. Conditions which are treatable by the composition of the present invention include, without limitation, narcolepsy, obstructive sleep apnea/hypopnea and sleepiness in shift workers, idiopathic hypersomnia, multiple sclerosis-induced fatigue; post-anaesthesia grogginess; associated fatigue and opioid-induced sedation; fatigue in Charcot-Marie-Tooth Disease and everyday cat-napping, and for reduction or prevention of sleepiness throughout a desired period.

According to some embodiments, sildenafil is released during or before a period of desired sleeping, while said modafinil is released at or near or toward the period of desired wakefulness; or alternatively wherein said sildenafil and said modafinil are released simultaneously or substantially simultaneously at or near or toward a period of desired wakefulness.

Compositions of the present invention may, if desired, be presented in a pack or dispenser device, such as an FDA approved kit, which may contain one or more dosage forms containing the active ingredient. The pack may, for example, comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

According to some embodiments, any of the compositions of the present invention may optionally further comprise an additional active ingredient, such as, for example, an agent for reducing or eliminating headache, such as an NSAID or paracetamol. The additional active ingredient may be present, for example, in the core, or in one or more of the coating layers.

Additional advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

The Examples given below are intended only as illustrations of various embodiments Of the present invention, and are not intended to be limiting in any way.

### Example 1

### Uncoated Modafinil Formulations

### Core Compositions #1-6

Cores having the following compositions were prepared:

TABLE 1A

| | 1 | 11-25 | 2 | 11-27/A | 3 | 11-27/B | 4 | 11-27/C | 5 | 11-95 |
|---|---|---|---|---|---|---|---|---|---|---|
| | % tablet | Weight | % tablet | Weight | % tablet | Weight | % tablet | Weight | % tablet | Weight |
| Materials | core | mg/tab | core | mg/tab | core | mg/tab | core | mg/tab | core | mg/tab |
| Modafinil | 16.7 | 100 | 18.2 | 100 | 18.2 | 100 | 18.2 | 100 | 16.7 | 100 |
| Lactose Monohydrate | 16.6 | 100 | 18.2 | 100 | — | — | 9.1 | 50 | — | — |
| Starch 1500 | 3 | 18 | — | — | — | — | — | — | — | — |
| Croscarmellose Sodium | 2 | 12 | 2 | 11 | 2 | 11 | 2 | 11 | 1.8 | 10.8 |
| Microcrystalline cellulose PH 101 | — | — | — | — | 18.2 | 100 | 9.1 | 50 | 14.7 | 88.4 |
| Povidone K 30 | 2 | 12 | 1.1 | 6.3 | 1.1 | 6.3 | 6.3 | 1.15 | 2.8 | 16.8 |
| Microcrystalline cellulose PH 102 | 59.2 | 355 | 58 | 318.9 | 58 | 318.9 | 318.9 | 58 | 62.4 | 374.5 |
| Tween 80 | — | — | — | — | — | — | — | — | 0.2 | 1.1 |
| Silicon dioxide colloidal | — | — | 2 | 11 | 2 | 11 | 2 | 11 | 0.9 | 5.4 |
| Magnesium stearate | 0.5 | 3 | 0.5 | 2.8 | 0.5 | 2.8 | 0.5 | 2.8 | 0.5 | 3 |
| Total Core | 100 | 600 | 100 | 550 | 100 | 550 | 100 | 550 | 100 | 600 |

| | 6 | 11-1-37 |
|---|---|---|
| | % tablet | Weight |
| Materials | core | mg/tab |
| Modafinil | 16.7 | 100 |
| Microcrystalline cellulose PH 101 | 15.6 | 93.7 |
| Povidone K 30 | 1.1 | 6.3 |

TABLE 1B

| | 11-1-65, 67, 73 . . . | |
|---|---|---|
| Materials | % tablet core | Weight mg/tab |
| Modafinil | 16.7 | 100 |
| Crospovidone | 1.8 | 10.5 |
| Microcrystalline cellulose PH 101 | 15.6- | 93.7 |
| Povidone K 30 | 1.1 | 6.3 |
| Microcrystalline cellulose PH 102 | 53.5 | 321.1 |
| Crospovidone | 10 | 60.0 |
| Silicon dioxide colloidal | −0.9 | 5.5- |
| Magnesium stearate | 0.5 | 3 |
| Total Core | 100 | 600 |

The cores of Modafinil, 100 mg tablets, were composed of a granulate which included: Modafinil, Lactose Monohydrate or Microcrystalline Cellulose PH 101 (filler), Povidone K 30 (binder), Croscarmellose Sodium and Starch 1500 or Crospovidone as disintegrant.

The granulates were prepared by a wet granulation process using a low shear wet granulator, followed by vacuum drying and sieving of the resulting granules. Ethanol was used as a granulating solvent. The granules were further mixed with other excipients, including: Microcrystalline cellulose PH 102 as filler, Tween 80 as release controlling agent, Croscarmellose Sodium or Crospovidone as disintegrant, Silicon dioxide as a glidant and magnesium stearate as lubricant.

The granulation process was performed in a Mini-V-Processor. Granulate was sieved through 812 micron sieve, and then blended for 10 minutes with a mixture of Crospovidone and Silicon dioxide, which was previously sieved through a 812 micron sieve. Microcrystalline cellulose PH 102 was then added and the mixture was blended for 30 minutes. Magnesium stearate, which was previously sieved (through a 600 micron screen sieve), was then added into the mixture and blended for additional 3 minutes. The tabletting mixture was then compressed with a MINI KORSH tabletting press equipped with a suitable punches caplet (8.2×14.5 mm).

### Release Profile of Uncoated Modafinil Cores

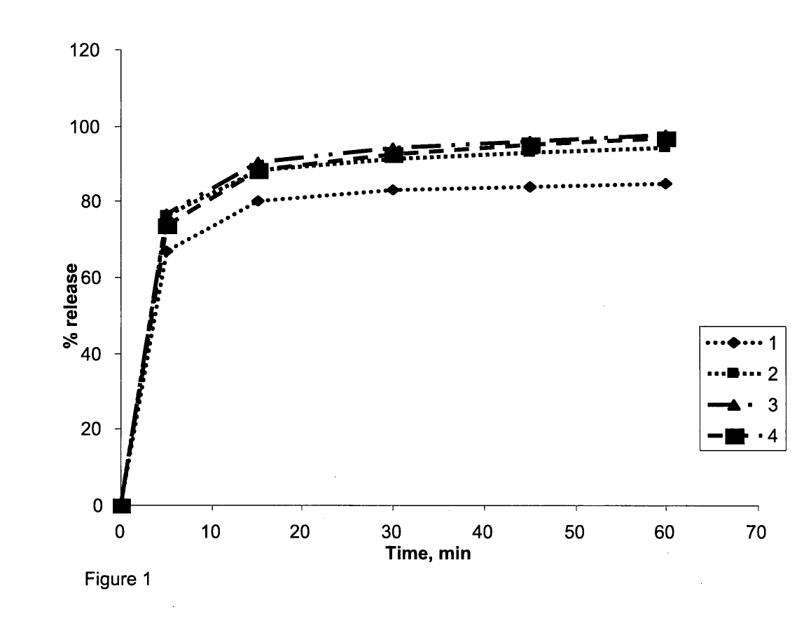

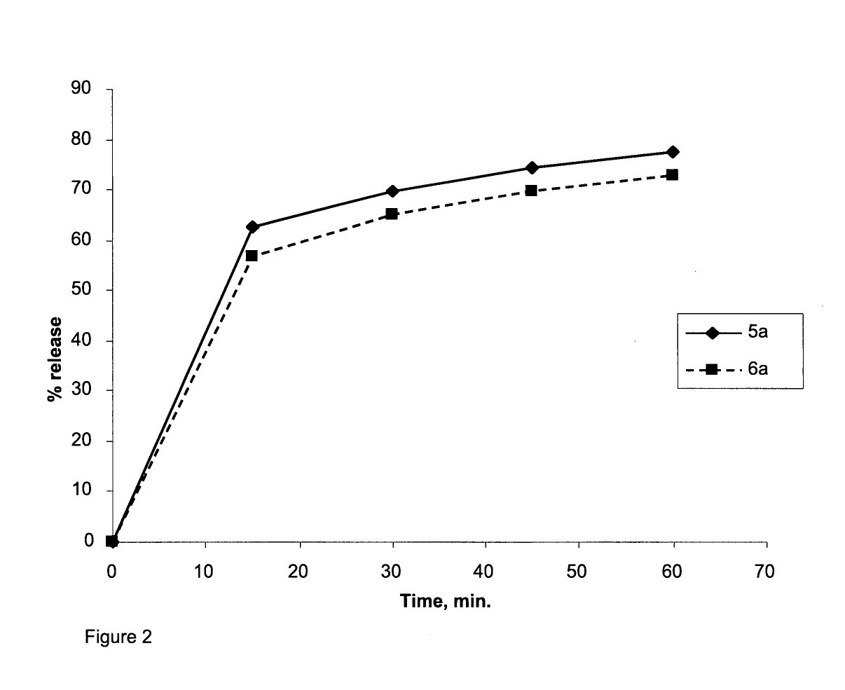

The release profiles of the uncoated cores were measured in order to demonstrate the effect of the one or more coatings of the present invention. Dissolution tests were performed in apparatus type 2 (paddles), at 37° C., 50 rpm, Medium: 900 ml buffer: Dissolution tests were performed in apparatus type 2 (paddles), at 37° C., 50 rpm, Medium: 900 ml buffer: 0.1N HCL (core formulations 1-4) and in buffer USP SIF pH=6.8 (core formulations 5 and 6). The results were analyzed using an automatic UV detector. The results are summarized in Tables 2 and 3 and graphically demonstrated in FIGS. 1 and 2.

TABLE 2

| Time, | % Release | | | |
|---|---|---|---|---|
| min | #1 | #2 | #3 | #4 |
| 0 | 0 | 0 | 0 | 0 |
| 5 | 67 | 76.3 | 76.7 | 73.7 |
| 15 | 80 | 88.5 | 90.3 | 88.1 |
| 30 | 83 | 91.5 | 94.2 | 92.4 |
| 45 | 84 | 93.1 | 96.2 | 95 |
| 60 | 85 | 94.1 | 97.7 | 96.8 |

TABLE 3

| Time, | % Release | |
|---|---|---|
| min | 5a | 6a |
| 0 | 0 | 0 |
| 15 | 62.6 | 56.7 |
| 30 | 69.7 | 65.1 |
| 45 | 74.2 | 69.7 |
| 60 | 77.3 | 72.7 |

FIG. 1 shows the dissolution of uncoated cores (1-4) in 0.1N HCL (average release of 6 tablets). FIG. 2 shows the dissolution of uncoated cores (5 and 6) in buffer phosphate pH=6.8 (average release of 6 tablets).

### Example 2

### Coated Modafinil Compositions

### Coating Composition

The core formulations of Example 1 were each coated with either a single layer of TCDS coating, or with two coating layers (a subcoating layer and a TCDS coating). Delay time (lag time) of release of active material was controlled by modifying the weight of the TCDS coating layer.

For these examples, which are provided for illustrative purposes only, and are not intended to be limiting, the subcoating layer comprised Povidone K 30 as a coating polymer and microcrystalline cellulose (Avicel PH 101) as a glidant.

Povidone K-30 was dissolved in ethanol to obtain a clear solution. Microcrystalline cellulose PH 101 was added and stirred to obtain a homogeneous suspension. The resulting suspension was stirred throughout the whole coating process.

The coating of cores was performed in a perforated pan coater using a spraying pressure of 0.5-1.1 Bar at 35° C. The coated tablets were dried in the coater at 45° C. for about 15 minutes.

The TCDS layer, which is a diffusion controlling layer, comprised ethylcellulose as a coating polymer and Avicel PH 102 as a diffusion controlling agent.

Ethylcellulose was dissolved in ethanol to obtain a clear solution (4% w/w). Cetyl alcohol and sodium lauryl sulphate were then added, and stirred to obtain a clear solution.

Microcrystalline cellulose PH 102 was added and stirred to obtain a homogeneous suspension. The resulting suspension was stirred throughout the whole coating process.

The coating of tablets was performed in a perforated pan coater (Dumoulin Machine) using a spraying pressure of 1.5-2.6 Bar at 55° C. The coated tablets were dried in an oven at 60° C. for about 16 hours.

Core formulation #6 of Example 1 was coated with either a single TCDS coating, or with a subcoating and TDS coating.

The subcoating was as follows:

| | Gram/coating | % dry weight | mg/tablet |
|---|---|---|---|
| Ethanol | 30 | — | — |
| PVP K-30 | 3 | 50% | 3.0 |
| Avicel PH 101 | 3 | 50% | 3.0 |
| Total dry components | 6 | 100.0% | 6.0 |

The TCDS coating was as follows:

| | | | #1 |
|---|---|---|---|
| | Gram/coating | % dry weight | mg/tablet |
| Ethanol | 12000 | — | — |
| Ethocel 20 | 533 | 37.9 | 34.17 |
| Avicel PH 102 | 790 | 60.4% | 50.64 |
| Cetyl Alcohol | 53 | 3.8% | 3.4 |
| Sodium Lauryl Sulphate | 28 | 2% | 1.8 |
| Total dry components | 1404 | 100.0% | 90.1 |

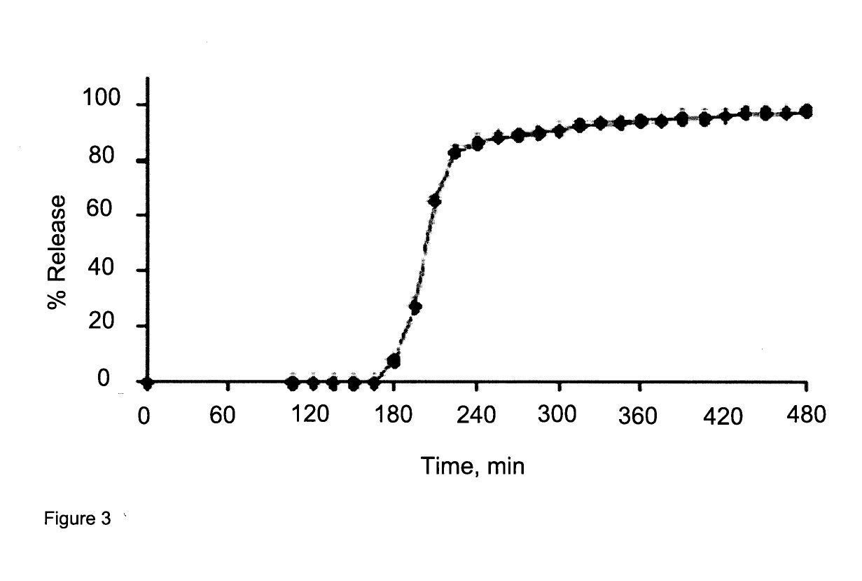

The dissolution results for the TCDS coated core #6 are summarized in Table 4. FIG. 3 shows a release profile of TCDS core 1 in buffer phosphate pH=6.8 (Average Release of 6 tablets).

TABLE 4

| Time, min | % Release |
|---|---|
| 0.0 | 0 |
| 105.0 | 0 |
| 120.0 | 0 |
| 135.0 | 0 |
| 150.0 | 0 |
| 165.0 | 0 |
| 180.0 | 8 |
| 195.0 | 27 |
| 210.0 | 66 |
| 225.0 | 83 |
| 240.0 | 87 |
| 255.0 | 89 |
| 270.0 | 90 |
| 285.0 | 91 |
| 300.0 | 91 |
| 315.0 | 93 |
| 330.0 | 94 |
| 345.0 | 94 |
| 360.0 | 95 |
| 375.0 | 95 |
| 390.0 | 96 |
| 405.0 | 96 |
| 420.0 | 97 |
| 435.0 | 97 |
| 450.0 | 98 |
| 465.0 | 98 |
| 480.0 | 98 |

### Example 3

### Coated Modafinil Compositions

### Coating Compositions

As a further example, all core types were coated with either a TCDS layer alone or a TCDS layer with a subcoating, as follows:

| | #1 | #2 | #5A | #6 A | #6 B | #6 C |
|---|---|---|---|---|---|---|
| | mg/tab. | mg/tab. | mg/tab. | mg/tab. | mg/tab. | mg/tab. |
| Povidone K 30 | 3.0 | 3.0 | | | | |
| Microcrystalline cellulose PH 101 | 3.0 | 3.0 | | | | |
| Total Protective coat | 6 | 6 | | | | |
| Ethyl cellulose N 22 | | | 21.3 | 34.1 | 34.2 | 38.0 |
| Microcrystalline cellulose PH 102 | | | 31.5 | 50.5 | 50.6 | 56.3 |
| Cetyl Alcohol | | | 2.1 | 3.4 | 3.4 | 3.8 |
| Sodium Lauryl Sulphate | | | 1.1 | | 1.8 | 2.0 |
| Total TCDS coat | | | 56 | 88 | 90 | 100 |

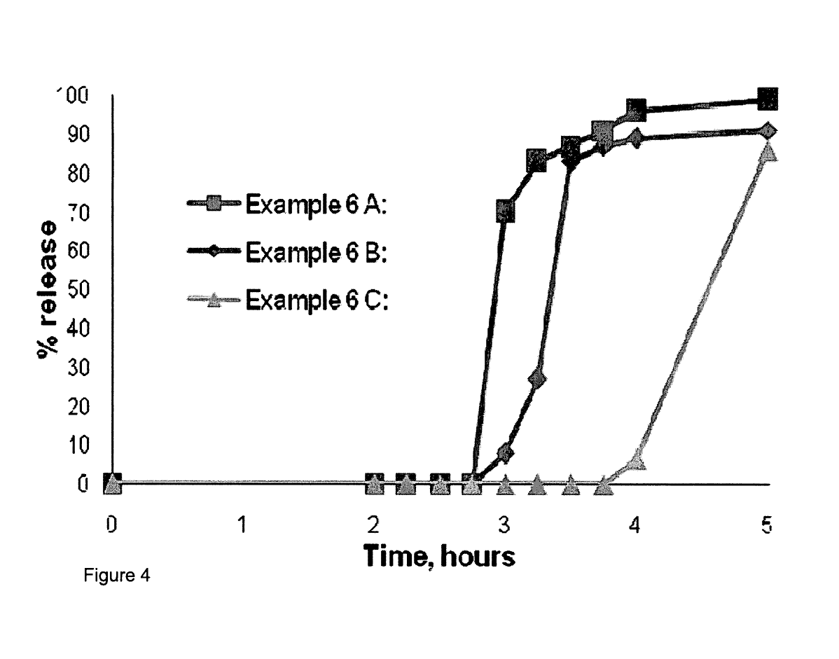

The coating layers were applied as described above for Example 2. Dissolution tests were performed as described above for Example 1. Results are shown in Tables 5 (coated composition 5A) and 6 (coated compositions 6A, 6B and 6C), and in FIG. 4 (coated compositions 6A, 6B and 6C).

TABLE 5

| Time, | #5 A: |
|---|---|
| hours | % Release |
| 0 | 0 |
| 2 | 0 |
| 2.25 | 21 |
| 2.5 | 25 |
| 2.75 | 44 |
| 3 | 65 |
| 3.25 | 70 |
| 3.5 | 74 |
| 3.75 | |
| 4 | 77 |
| 5 | |

TABLE 6

| Time, | #6 A: | #6 B: | #6 C: |
|---|---|---|---|
| hours | % Release | % Release | % Release |
| 0 | 0 | 0 | 0 |
| 2.5 | 0 | 0 | 0 |
| 2.75 | 0 | 0 | 0 |
| 3 | 70 | 8 | 0 |
| 3.25 | 83 | 27 | 0 |
| 3.5 | 87 | 83 | 0 |
| 3.75 | 91 | 87 | 0 |
| 4 | 96 | 89 | 6.6 |
| 5 | 99 | 91 | 86 |

### Example 4

### Multilayered Modafinil Coated Formulations

Tablet cores #4 and #5 of Example 1 were coated with 2 layers of coating, comprising:

1. Subcoating layer: comprises Povidone K 30 as a coating polymer and microcrystalline cellulose PH 101 as a glidant; and

2. TCDS layer: diffusion controlling layer comprising ethyl cellulose as a coating polymer which is insoluble in aqueous medium and Avicel PH 102 as a diffusion controlling agent;

The coating formulations were as follows:

| | #4 P | #5 P |
|---|---|---|
| | mg/tab. | mg/tab. |
| Povidone K 30 | 1.0 | 1.0 |
| Microcrystalline cellulose PH 101 | 1.0 | 1.0 |
| Total Protective coat | 2 | 2 |
| Ethyl cellulose 20 | 13.1 | 13.1 |
| Microcrystalline cellulose PH 102 | 15.9 | 15.9 |
| Cetyl Alcohol | 1.4 | 1.4 |
| Sodium Lauryl Sulphate | 1.7 | 1.7 |
| Total TCDS coat | 32 | 32 |

For the protective coating layer, Povidone K-30 was dissolved in ethanol to obtain a clear solution. Microcrystalline cellulose PH 101 was added and stirred to obtain a homogeneous suspension. The resulting suspension was stirred throughout the whole coating process.

Coating of the cores was performed in a perforated pan coater at 35° C. The coated tablets were dried in the coater at 45° C. for about 15 minutes.

For the TCDS layer, ethyl cellulose was dissolved in ethanol to obtain a clear solution (4% w/w). Cetyl Alcohol and sodium lauryl sulphate were subsequently added and stirred to obtain a clear solution. Microcrystalline cellulose PH 102 was then added and stirred to obtain a homogeneous suspension. The resulting suspension was stirred throughout the whole coating process.

The coating of tablets was performed in a perforated pan coater at about 30-40° C. The coated tablets were dried in oven at 60° C. for about 16 hours.

Dissolution tests were performed in apparatus type 2 (paddles), at 37° C., 50 rpm, Medium: 900 ml buffer: buffer USP SIF without enzymes pH 6.8. The results were analyzed using automatic UV detector.

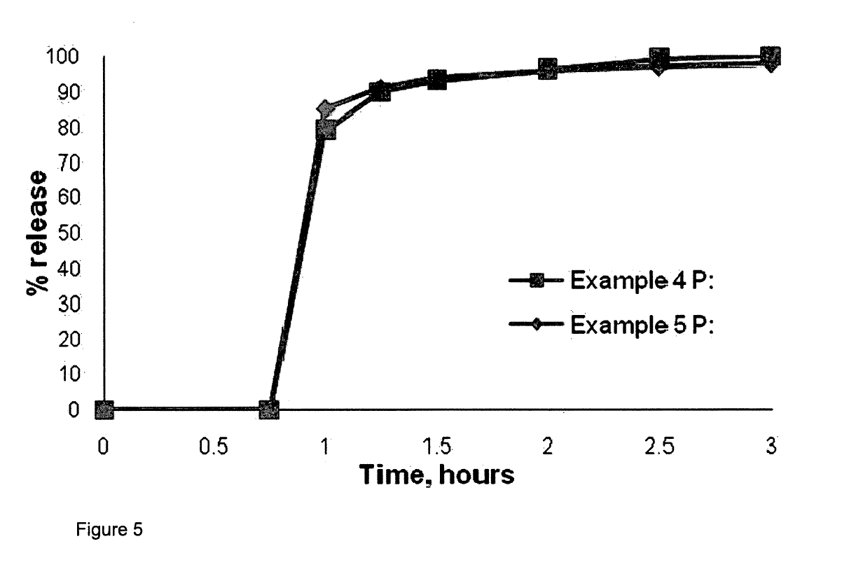

Results are presented in Table 7 and FIG. 5.

TABLE 7

| Time, | Example 4 P: | Example 5 P: |
|---|---|---|
| hours | % Release | % Release |
| 0 | 0 | 0 |
| 0.75 | 0 | 0 |
| 1 | 0 | 0 |
| 2.5 | 79 | 85 |
| 1.25 | 90 | 91 |
| 1.5 | 93 | 94 |
| 2 | 96 | 96 |
| 2.5 | 99 | 97 |
| 3 | 100 | 98 |

### Example 5

### Sildenafil Citrate Time Controlled Delayed Release Formulation

### Core Composition #7:

| Materials | % tablet core | Weight mg/tab |
|---|---|---|
| Sildenafil Citrate | 15.00 | 10.0% |
| Microcrystalline cellulose PH101 | 12.50 | 8.3% |
| Lactose | 8.50 | 5.7% |
| Crospovidone | 2.50 | 1.7% |
| Povidone K 30 | 1.50 | 1.0% |
| Microcrystalline cellulose PH102 | 87.10 | 58.1% |
| Crospovidone | 19.00 | 12.7% |
| Silica colloidal anhydrous | 3.00 | 2.0% |
| Magnesium Stearate | 0.90 | 0.6% |
| Total Core | 150.00 | 100.0% |

The cores of Sildenafil Citrate 15 mg tablets were composed of a granulate which included Sildenafil citrate, lactose monohydrate and microcrystalline cellulose PH 101 (fillers), Povidone K 30 (binder) and Crospovidone (as disintegrant).

The granulate was produced by a wet granulation process, using ethanol as a granulation solvent, followed by drying and sieving of the resulting granules through a 600 micron sieve. The granules were further mixed with other excipients, including microcrystalline cellulose PH 102 as filler, Crospovidone as disintegrant, silicon dioxide as glidant and magnesium stearate as lubricant. The tabletting mixture was then compressed with a WICK tabletting press equipped with suitable punches with diameter 7 mm.

### Release Profile of Uncoated Sildenafil Cores

Dissolution tests were performed in apparatus type 2 (paddles), at 37° C., 50 rpm, Medium: 900 ml buffer: 0.1N HCl or Phosphate buffer pH 6.8 (IF). The results were analyzed using automatic UV detector.

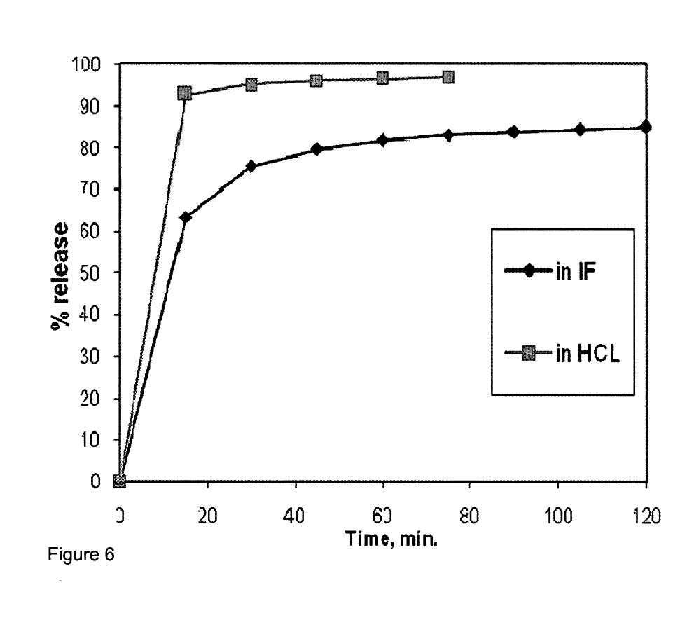

Results are presented in Table 8 and FIG. 6.

TABLE 8

| Time, | % Release | % Release |
|---|---|---|
| minutes | in IF | in HCL |
| 0 | 0 | 0 |
| 15 | 63.2 | 92.8 |
| 30 | 75.5 | 95.3 |
| 45 | 79.7 | 96.2 |
| 60 | 81.8 | 96.7 |
| 75 | 83.1 | 97.1 |
| 90 | 83.9 | |
| 105 | 84.5 | |
| 120 | 85 | |

### Example 6

### Coated Sildenafil Citrate Compositions

### Coating Layer:

Core composition #7 of Example 5 was coated with a Time Controlled Delivery System (TCDS) coating according to one of the following formulations:

| | 7 A -1 | 7 A-2 |
|---|---|---|
| Material: | mg/tablet | mg/tablet |
| Microcrystalline cellulose PH102 | 12.1 | 15.6 |
| Ethyl Cellulose N22 | 8.1 | 10.4 |
| Cethyl alcohol | 0.8 | 1.0 |
| Dry coating weight | 21.0 | 27.0 |

Ethyl cellulose N22 was dissolved in ethanol to obtain a clear solution (4% w/w). Cetyl alcohol was added and stirred to obtain a clear solution. Microcrystalline cellulose PH 102 was then added and stirred to obtain a homogeneous suspension. The resulting suspension was stirred throughout the whole coating process.

The coating of tablets was performed in a perforated pan coater at 30-40° C. The coated tablets were dried in an oven at 60° C. for about 16 hours.

### Release Profile of Coated Sildenafil Compositions

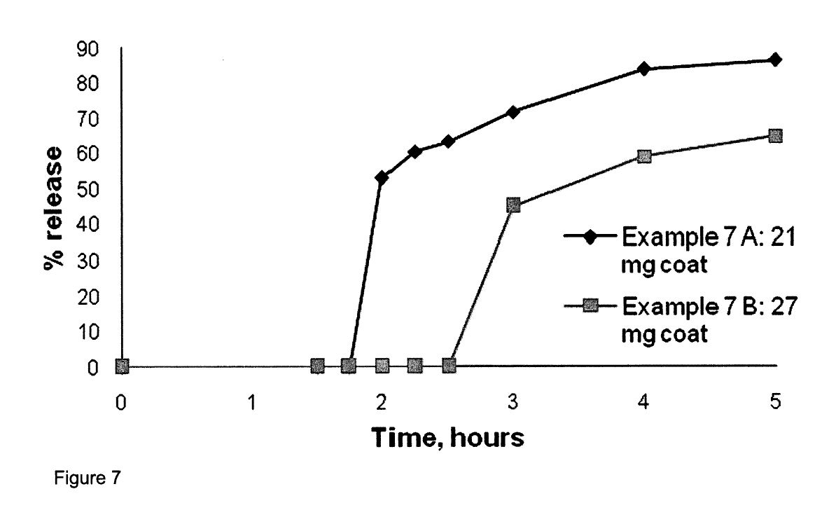

Dissolution tests were performed in apparatus type 2 (paddles), at 37° C., 50 rpm, Medium: 900 ml buffer: buffer USP SIF without enzymes pH 6.8. The results were analyzed using an automatic UV detector. Results are presented in Table 9 and FIG. 7.

TABLE 9

| Time, | % Release | % Release |
|---|---|---|
| hours | 21 mg coat | 27 mg coat |
| 0 | 0 | 0 |
| 1.5 | 0 | 0 |
| 1.75 | 0 | 0 |
| 2 | 53 | 0 |
| 2.25 | 60.4 | 0 |
| 2.5 | 63.2 | 0 |
| 3 | 71.6 | 44.9 |
| 4 | 83.8 | 58.9 |
| 5 | 86.4 | 64.8 |

### Example 7

### Multilayer Time Controlled Coating System for Sildenafil Citrate

### Core Composition:

| | Core #8 | | Core # 9 | |
|---|---|---|---|---|
| | % tablet | Weight | % tablet | Weight |
| Materials | core | mg/tab | core | mg/tab |
| Sildenafil Citrate | 15.0 | 23.1% | 21.07 | 32.4% |
| Microcrystalline cellulose PH101 | 12.0 | 19.2% | 10.00 | 15.4% |
| Crospovidone | 2.0 | 3.8% | 2.50 | 3.8% |
| Povidone K 30 | 1.0 | 2.3% | 1.50 | 2.3% |
| Microcrystalline cellulose PH102 | 31.8 | 48.9% | 28.17 | 43.3% |
| Silica colloidal anhydrous | 1.3 | 2.0% | 1.30 | 2.0% |
| Magnesium Stearate | 0.4 | 0.6% | 0.46 | 0.7% |
| Total Core | 65.0 | 100.0% | 65.00 | 100.0% |

The cores comprised granulate which included: Sildenafil citrate, microcrystalline cellulose PH 101 (filler), Povidone K 30 (binder) and Crospovidone as disintegrant. The granulate was produced by low shear wet granulation. Ethanol was used as a granulation solvent. The granulate was dried and sieved through 800 micron sieve. The granules were further mixed with other excipients, including microcrystalline cellulose PH 102 as filler, Crospovidone as disintegrant, Silicon dioxide as a glidant and magnesium stearate as lubricant. The tabletting mixture was then compressed using a Killian tabletting press equipped with suitable punches with diameter 5 mm.

### Coating:

Core compositions #8 and #9 were each coated with a multiple-layer coating. The delay time in active material release was controlled by adjusting the weight of TCDS coating layer.

The coatings were as follows:
1. Acid layer: a first inner coating layer comprising citric acid as drug release enhancer, Povidone K 30 as a coating polymer and silica colloidal anhydrous as a glidant;
2. TCDS layer: diffusion controlling layer comprising Eudragit E as a coating polymer which is soluble in acid aqueous medium and Avicel PH 105 as a diffusion controlling agent;
3. Protective coating layer: comprises Povidone K 30 as a coating polymer and microcrystalline cellulose (Avicel PH 105) as a glidant;
4. Enteric coating layer: comprises aetacrylic acid-ethyl acrylate copolymer as a coating polymer which is soluble in a aqueous medium with pH>5, triethyl citrate as a plasticizer and talc as a glidant
   Inner acid layer and TCDS layer coated tablets 8A, 8B, 9A, 9B:

| | Example # | | | |
|---|---|---|---|---|
| | 8 A | 8 B | 9 A | 9 B |
| | mg/ | mg/ | mg/ | mg/ |
| Materials: | tablet | tablet | tablet | tablet |
| Citric acid | 17.2 | 17.2 | 17.2 | 17.2 |
| Silica colloidal anhydrous | 0.9 | 0.9 | 0.9 | 0.9 |
| Povidone K 30 | 4.9 | 4.9 | 4.9 | 4.9 |
| Total Acid coating | 23.0 | 23.0 | 23.0 | 23.0 |
| Microcrystalline cellulose PH 105 | 6.4 | 8 | 7.2 | 8 |
| Eudragit E | 9.6 | 12 | 10.8 | 12 |
| Total TCDS coating | 16 | 20 | 18 | 20 |

For the acid layer, Povidone K-30 was dissolved in ethanol 85% to obtain a clear 1% w/w solution. Citric acid was added and dissolved. Colloidal silica was then added and stirred to obtain a homogeneous suspension. The resulting suspension was stirred throughout the whole coating process.

The coating of cores was performed in a perforated pan coater at 45° C. The coated tablets were dried in the coater at 45° C. for about 15 minutes.

For the TCDS layer, Eudragit E was dissolved in ethanol to obtain a clear solution (9.8% w/w). Microcrystalline cellulose PH 105 was then added and stirred to obtain a homogeneous suspension. The resulting suspension was stirred throughout the whole coating process.

The coating of tablets was performed in a perforated pan coater at about 35° C. The coated tablets were dried in an oven at 60° C. for about 16 hours.

Dissolution tests were performed in apparatus type 2 (paddles), at 37° C., 50 rpm, Medium: 900 ml buffer: Acetate buffer pH 5.8. The results were analyzed using automatic UV detector.

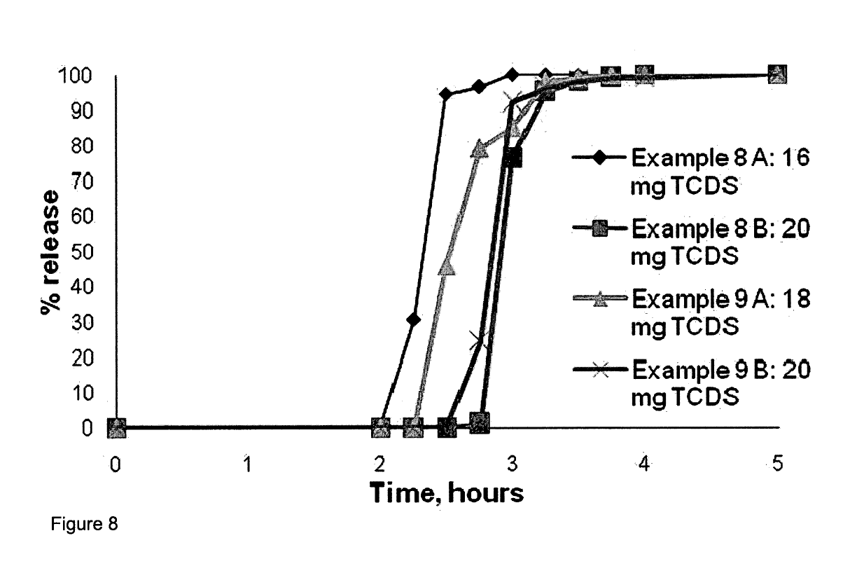

Results are presented in Table 10 and FIG. 8.

TABLE 10

| | Composition # | | | |
|---|---|---|---|---|
| | 8 A: 16 mg | 8 B: 20 mg | 9 A: 18 mg | 9 B: 20 mg |
| Time, | TCDS | TCDS | TCDS | TCDS |
| hours | % Release | % Release | % Release | % Release |
| 0 | 0 | 0 | 0 | 0 |
| 2 | 0 | 0 | 0 | 0 |
| 2.25 | 31 | 0 | 0 | 0 |
| 2.5 | 94 | 0 | 46 | 0 |
| 2.75 | 97 | 1 | 79 | 24 |
| 3 | 100 | 76 | 85 | 92 |
| 3.25 | 100 | 95 | 98 | 96 |
| 3.5 | 100 | 98 | 99 | 98 |
| 3.75 | 100 | 99 | 100 | 99 |
| 4 | 100 | 100 | 100 | 99 |
| 5 | 100 | 100 | 100 | 100 |

TCDS Coated Sildenafil Tablets 8C, 9C with Protective Coating Layer and Enteric Coating Layer:

Tablets 8B and 8C, described above, having an acidic inner layer and TCDS coating layer, were further coated with a protective layer on the TCDS layer, and subsequently by an enteric coating layer to protect the TCDS layer from low pH of gastric fluid.

The protective layer and enteric layer coating formulations were as follows:

| | Example # | |
|---|---|---|
| | 8 C | 9 C |
| | mg/tablet | mg/tablet |
| Protective coating | | |
| Povidone K 30 | 7.2 | 7.2 |
| Microcrystalline cellulose PH 105 | 0.8 | 0.8 |
| Total Protective coating | 8 | 8 |
| Enteric coating | | |
| Methacrylic acid-ethyl acrylate copolymer | 4.4 | 5.0 |
| Talc | 2.2 | 2.5 |
| Triethyl citrate | 0.4 | 0.5 |
| Total Enteric coating | 7 | 8 |

For the protective coating layer, Povidone K-30 was dissolved in ethanol 96% to obtain a clear 15% w/w solution. Microcrystalline cellulose PH 105 was then added and stirred to obtain a homogeneous suspension. The resulting suspension was stirred throughout the whole coating process.

The coating was performed in a perforated pan coater at about 45° C. The coated tablets were dried in the coater at 45° C. for about 15 minutes.

For the enteric coating layer, triethyl citrate was first dispersed at high speed in purified water (6% w/w). Talc was then added and stirred to obtain a homogeneous suspension. The resulting suspension was mixed with 30% aqueous dispersion of methacrylic acid-ethyl acrylate copolymer and stirred throughout the whole coating process.

The coating of tablets was performed in a perforated pan coater at about 35° C. The coated tablets were dried in oven at 40° C. for about 16 hours.

### Release Profile of Coated Sildenafil Tablets 8C and 9C

Dissolution tests were performed in apparatus type 2 (paddles), at 37° C., 50 rpm, Medium: 900 ml buffer: 1 hour in HCL 0.1N, than Acetate buffer pH 5.8. The results were analyzed using automatic UV detector.

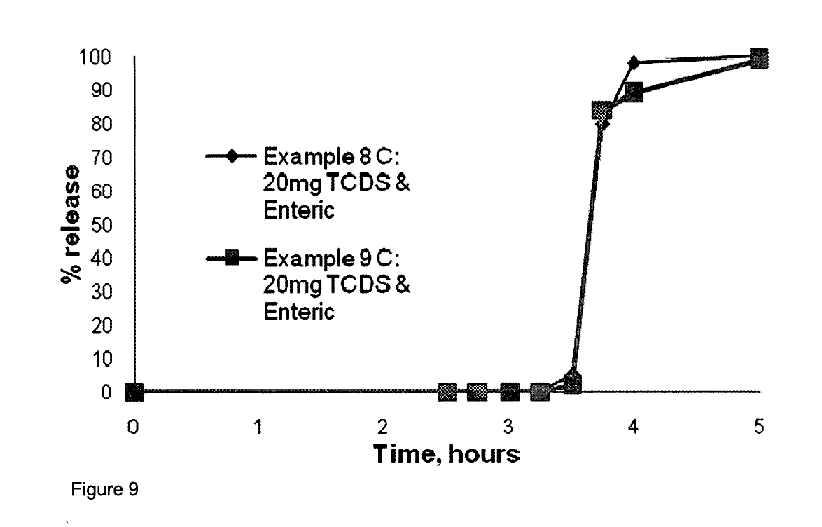

Results are presented in Table 11 and FIG. 9.

TABLE 11

| | 8 C: 20 mg | 9 C: 20 mg |
|---|---|---|
| Time, | TCDS & Enteric | TCDS & Enteric |
| hours | % Release | % Release |
| 0 | 0 | 0 |
| 2.5 | 0 | 0 |
| 2.75 | 0 | 0 |
| 3 | 0 | 0 |
| 3.25 | 0 | 0 |
| 3.5 | 5 | 2 |
| 3.75 | 80 | 84 |
| 4 | 98 | 89 |
| 5 | 100 | 99 |

### Multilayer Coated Sildenafil TCDS Coated Tablets 9D:

Acid layer and TCDS layer coated tablets 9A were coated with protective and enteric coating layers to protect the TCDS layer from the low pH of gastric fluid. The coating formulation was as follows:

| | Example # |
|---|---|
| | 9 D |
| Materials: | mg/tablet |
| Povidone K 30 | 7.2 |
| Microcrystalline cellulose PH 105 | 0.8 |
| Total Protective coating | 8 |
| Methacrylic acid-ethyl acrylate copolymer | 5.0 |
| Talc | 2.5 |
| Triethyl citrate | 0.5 |
| Total Enteric coating | 8 |

The protective and enteric coating layers were prepared and applied as described above for 8C and 9C.

### Release Profile of Multilayer Coated Sildenafil Tablet 9D:

Dissolution tests were performed in apparatus type 2 (paddles), at 37° C., 50 rpm, Medium: 900 ml buffer: 1 hour in HCL 0.1N, then phosphate buffer pH 6.8. The results were analyzed using automatic UV detector.

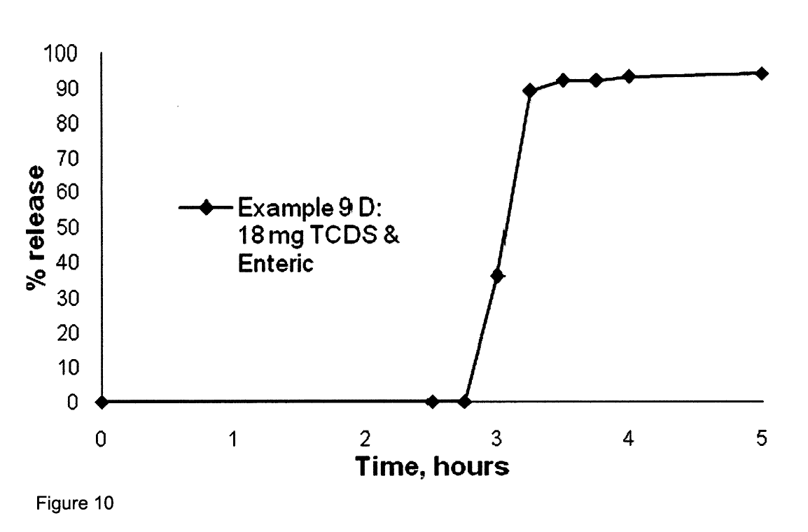

Results are presented in Table 12 and FIG. 10.

TABLE 12

| | Example 9 D: 18 mg |
|---|---|
| Time, | TCDS & Enteric |
| hours | % Release |
| 0 | 0 |
| 2.5 | 0 |
| 2.75 | 0 |
| 3 | 36 |
| 3.25 | 89 |
| 3.5 | 92 |
| 3.75 | 92 |
| 4 | 93 |
| 5 | 94 |

### Example 8

### Combination Modafinil and Sildenafil Citrate Delayed Release Formulation

One each of tablets 6A of Example 3, and 9D of Example 7 were packaged in a hard gelatin capsule.

Dissolution tests were performed in apparatus type 2 (paddles), at 37° C., 50 rpm, Medium: 900 ml buffer: 1 hour in HCL 0.1N, than Phosphate buffer pH 6.8. The results were analyzed using automatic UV detector.

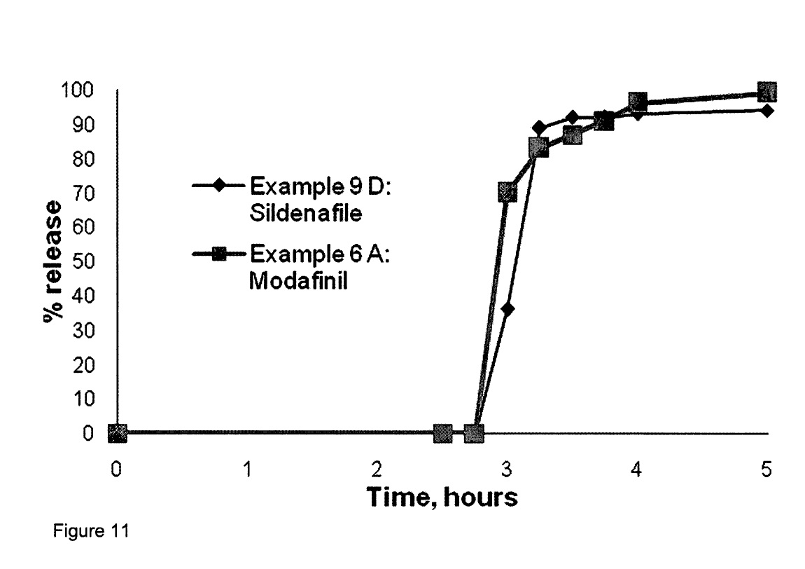

Results are presented in Table 13 and FIG. 11.

TABLE 13

| Time, | 9 D: Sildenafil | 6 A: Modafinil |
|---|---|---|
| hours | % Release | % Release |
| 0 | 0 | 0 |
| 2.5 | 0 | 0 |
| 2.75 | 0 | 0 |
| 3 | 36 | 70 |
| 3.25 | 89 | 83 |
| 3.5 | 92 | 87 |
| 3.75 | 92 | 91 |
| 4 | 93 | 96 |
| 5 | 94 | 99 |

### Bioavailability Study

A randomized, pharmacokinetic pilot study is undertaken to evaluate the bioavailability of test formulations of the combination dosage form of low-dose modafinil and low-dose sildenafil. For the study, tablets are prepared according to any suitable example above.

### Efficacy Study

The formulation of the present invention is believed to have increased efficacy and to be capable of providing at least similar, if not greater, pharmaceutical effects with the active ingredients of the synergistic combination to those of formulations known in the art for each active ingredient alone. Preferably the formulation provides a significantly decreased dosage amount as compared to orally administered formulations that are known in the art for each active ingredient separately. Without wishing to be limited by a single hypothesis, it is also possible that lower side effects may be observed with the formulation of the present invention, again as compared to other orally administered dosage forms that are known in the art.

Optionally, such a decreased dosage amount of the combined active ingredients, preferably modafinil and sildenafil, comprises up to about 60% of the regular dosage amount, more preferably up to about 50% and most preferably up to about 40% of the regular dosage amount for each active ingredient separately. Optionally, the dosage amount comprises up to about 30% of the regular dosage amount. One non-limiting example of a “regular” dosage amount is that administered with each currently available reference product for each of modafinil and sildenafil separately. The dosage amount during a 24 hour period is also determined by the dosage frequency; preferably, the formulation of the present invention is not administered more frequently than the “regular” orally administered formulations; more preferably, the formulation of the present invention is administered once daily, optionally in the evening.

A clinical study investigates the issue of both pharmaceutical efficacy as well as bioavailability. This study compares the efficacy and pharmacokinetic parameters of a tablet according to the present invention, with the reference products (also as used in the bioavailability studies above) which each contain a regular, separate dosage of each of the two drugs. The clinical study is conducted with patients suffering from one or more of a requirement for increased wakefulness, a requirement for reduction or prevention of sleepiness throughout a desired period, jet lag, sleepiness in shift workers, sleepiness during the first hours post awakening, morning awakening, sleep apnea and/or narcolepsy, although it should be noted that this is for the purpose of the study only and is not intended to be limiting in any way.

The primary end point criteria of the study is equivalent or superior mean percent reductions from baseline (i.e. before the study) in sleepiness or drowsiness observed in patients taking the tablet according to the present invention, as compared to the reference products. The study also preferably demonstrates synergism of the combined formulation. Preferably, one group of patients receives one reference product, while another group receives the second reference product; yet a third group preferably receives the combination formulation of the present invention.

All three sets of patients take one tablet per day (present invention or reference) in the evening. Each set includes 80 patients suffering from one of the conditions to be treated. The patients either have not been previously treated with either of the two active ingredients, or are undergoing a 6 week washout period (during which no drug is given) before the study begins. The study is double-blind, randomized and multicenter. Any potential adverse effects are detected with clinical and laboratory testing.

A treatment period of 6 weeks occurs, with periodic measurements of the blood.

As noted above, the clinical study shows that the tablet of the present invention (preferably with the lower dosage amount per tablet) is at least as pharmaceutically effective as the immediate release reference product, thereby providing at least similar clinical efficacy but with a significantly lower dose (up to about 40% of the immediate release reference product).

Treatment with the tablet of the present invention has at least similar effects as in published literature/studies, although the final comparison is made with the set of patients who are taking the reference product within the study itself.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A composition comprising:
a core comprising at least one of a wakefulness-inducing agent and a phosphodiesterase type 5 inhibitor as active ingredient and;
a time-controlled delivery system (TCDS) coating, wherein said TCDS coating controls release of said active ingredient in said core as a delayed onset, slow release formulation.

2. The composition of claim 1, wherein said wakefulness-inducing agent is selected from the group consisting of modafinil, armodafinil, a xanthine derivative, caffeine, a centrally acting sympathomimetic, or a combination thereof.

3. The composition of claim 1, wherein said phosphodiesterase type 5 inhibitor is selected from the group consisting of sildenafil, tadalafil, vardenafil or a combination thereof.

4. The composition of claim 1, wherein said core further comprises at least one of a filler, a binder, a disintegrant, a release controlling agent, a glidant and a lubricant.

5. The composition of claim 1, wherein said TCDS coating is selected from the group consisting of a pH dependent coating film; a combination of two or more water insoluble polymers; a combination of at least one swellable polymer and at least one water insoluble polymer; a flexible but non-soluble coating; a swellable coating; and a water permeable coating.

6. The composition of claim 5, further comprising a diffusion controlling agent.

7. The composition of claim 5, wherein said pH dependent polymer is selected from the group consisting of a hydroxypropylmethyl cellulose phthalate, polyvinyl acetate phthalate, cellulose acetate phthalate, hydroxypropylmethyl cellulose acetate succinate, poly(methacrylic acid, methyl methacrylate) 1:1 and poly(methacrylic acid, ethyl acrylate) 1:1, alginic acid, and sodium alginate.

8. The composition of claim 5, wherein said polymer comprises Eudragit™.

9. The composition of claim 5, wherein said water insoluble polymer is selected from the group consisting of a polymethylaminoethylacrylate/ethylmethacrylate copolymer; an ethylmethacrylate/chlorotrimethylammoniumethyl methacrylate copolymer; a dimethylaminoethylmethacrylate/methylmethacrylate and butylmethacrylate copolymer; a copolymer based on neutral methacrylic acid esters and dimethylaminoethyl methacrylate esters; an ethylacrylate and methylacrylate/ethylmethacrylate and methyl methylacrylate copolymer; ethylcellulose; shellac; zein; a wax; paraffin; cellulose acetate; cellulose propionate; cellulose acetate; propionate; cellulose acetate butyrate; cellulose acetate phthalate; cellulose triacetate; poly(methyl methacrylate); poly(ethylmethacrylate); poly(butyl methacrylate); poly(isobutyl methacrylate); poly(hexyl methacrylate); poly (isodecyl methacrylate); poly(lauryl methacrylate); poly(phenyl methacrylate); poly (methylacrylate); poly(isopropyl acrylate); poly(isobutyl acrylate); poly(octadecyl acrylate); poly(ethylene); poly(ethylene oxide); poly(ethyleneterephthalate); poly (vinyl isobutyl ether); poly(vinyl acetate); poly(vinyl chloride) and polyurethane, or mixtures thereof.

10. The composition of claim 9, wherein said water insoluble polymer comprises ethylcellulose.

11. The composition of claim 5, further comprising a plasticizer.

12. The composition of claim 1, further comprising a protective subcoating layer between said core and said TCDS coating.

13. The composition of claim 1, further comprising an enteric coating on said TCDS coating.

14. The composition of claim 13, further comprising a protective subcoating layer between said TCDS coating and said enteric coating.

15. The composition of claim 12, wherein said protective subcoating layer comprises a coating polymer selected from the group consisting of polyvinyl pyrrolidone, hydroxypropyl cellulose, hydroxypropyl methylcellulose, methyl cellulose, ethylcellulose, and gelatin polyethylene oxide.

16. The composition of claim 15, wherein said coating polymer comprises polyvinyl pyrrolidone.

17. The composition of claim 15, wherein said protective subcoating layer further comprises a glidant.

18. The composition of claim 17, wherein said glidant is selected from the group consisting of microcrystalline cellulose, talc, colloidal hydrated aluminum silicate, and silicon dioxide.

19. The composition of claim 1 further comprising an acidic coating layer between said core and said TCDS layer.

20. The composition of claim 19, wherein said acidic coating layer comprises an acidifying agent and a coating polymer.

21. The composition of claim 20, wherein said acidifying agent comprises citric acid and said coating polymer comprises Povidone.

22. The composition of claim 20, wherein said acidic coating layer further comprises a glidant.

23. The composition of claim 1, wherein said active ingredient comprises a wakefulness-inducing agent and a phosphodiesterase type 5 inhibitor.

24. The composition of claim 23, wherein said a wakefulness-inducing agent comprises modafinil or pharmaceutically acceptable salts thereof, and said phosphodiesterase type 5 inhibitor comprises sildenafil, or pharmaceutically acceptable salts thereof.

25. The composition of claim 24, wherein one or both of said modafinil and said sildenafil are present in a lower than normal dose.

26. The composition of claim 25, wherein said lower than normal dose is a sub-therapeutic dose.

27. The composition of claim 24, wherein said core comprises an active core.

28. The composition of claim 24, further comprising an active coating layer.

29. The composition of claim 28, wherein said active core comprises a first of modafinil or sildenafil, and said active coating layer comprises a second of modafinil or sildenafil.

30. The composition of claim 29, wherein said sildenafil is released during or before the period of desired sleeping, while said modafinil is released at or near or toward the period of desired wakefulness; or alternatively wherein said sildenafil and said modafinil are released simultaneously or substantially simultaneously and/or at or near or toward the period of desired wakefulness.

31. A method of achieving one or more of increasing wakefulness, treatment or prevention of sleepiness, treatment or prevention of jetlag, treatment or prevention of sleep disorders associated with excessive sleepiness, and reduction or prevention of sleepiness throughout a desired period, comprising administering a composition comprising a core comprising at least one of a wakefulness-inducing agent and a phosphodiesterase type 5 inhibitor as active ingredient and a time-controlled delivery system (TCDS) coating.

32. The method of claim 31, wherein said sleep disorder is selected from the group consisting of narcolepsy, obstructive sleep apnea/hypopnea and sleepiness in shift workers, idiopathic hypersomnia, multiple sclerosis-induced fatigue; post-anaesthesia grogginess; opioid-induced sedation; and fatigue in Charcot-Marie-Tooth Disease.

33. The composition of claim 1, further comprising an additional active ingredient.

34. The composition of claim 33, wherein said additional active ingredient comprises an agent for reducing or eliminating headache.

35. The composition of claim 34, wherein said agent comprises one or more of an NSAID and paracetamol.

36. A synergistic dosage form for treating sleep related disorders, comprising a synergistic combination of a PDE5 inhibitor and a wakefulness inducing agent.
